(19) United States
(12) Patent Application Publication
(10) Pub. No.: US 2026/0174391 A1
(43) Pub. Date: Jun. 25, 2026

(54) BIOLOGICAL SIGNAL ACQUISITION DEVICE

(71) Applicants: DENSO CORPORATION, Kariya-city (JP); TOYOTA JIDOSHA KABUSHIKI KAISHA, Toyota-shi (JP); MIRISE Technologies Corporation, Nisshin-shi (JP)

(72) Inventors: Yuki ANNO, Nisshin-shi (JP); Yoshinori Tsuchiya, Nisshin-shi (JP)

(21) Appl. No.: 19/350,438

(22) Filed: Oct. 6, 2025

(30) Foreign Application Priority Data
Dec. 20, 2024 (JP) 2024-225324

(51) Int. Cl. A61B 5/00; A61B 5/024

(52) U.S. Cl. CPC A61B 5/6893; A61B 5/02444; A61B 5/7203; A61B 2562/04

(57) ABSTRACT

A biological signal acquisition device is provided for detecting biological signals from a human body seated on a seat. The device includes a sensor that detects the biological signal and a noise reduction member positioned between the sensor and the seat, in contact with both. The noise reduction member reduces noise transmitted from the seat to the sensor. The device also includes at least one of a circuit or a processor with a memory storing computer program code, which processes the detected biological signal to obtain biological information. The noise reduction member has a projected area larger than that of the sensor in an opposing direction, and its Young's modulus is higher than that of the seat's contact portion, thereby improving signal accuracy by minimizing noise interference.

### CROSS REFERENCE TO RELATED APPLICATION

The present application is based on Japanese Patent Application No. 2024-225324 filed on Dec. 20, 2024, the entire disclosure of which is incorporated herein by reference.

### TECHNICAL FIELD

The present disclosure relates to a biological signal acquisition device.

### BACKGROUND

Conventional biological signal acquisition devices have sensors, such as piezoelectric sensors, that are placed in seats of vehicles. These sensors acquire biological signals, such as ballistocardiograms, heartbeat-induced body movement, and cardiac-induced body vibrations, from a person seated on the cushion.

### SUMMARY

According to at least one embodiment, a biological signal acquisition device includes a sensor that detects a biological signal of a human body in a seated state where the human body is seated on a seat. The device has a noise reduction member disposed between the sensor and the seat, in contact with both the sensor and the seat, and the noise reduction member reduces noise input from the seat to the sensor. The device further includes at least one of (i) a circuit and (ii) a processor with a memory storing computer program code executable by the processor, and the at least one of the circuit and the processor obtains biological information using the biological signal detected by the sensor. An opposing direction is defined as a direction along which the sensor, the noise reduction member, and a contact portion of contact between the noise reduction member and the seat are aligned. A projected area of the noise reduction member may be larger than a projected area of the sensor when viewed in the opposing direction, and a Young's modulus of the noise reduction member may be higher than a Young's modulus of the contact portion of the seat.

### BRIEF DESCRIPTION OF THE DRAWINGS

The details of one or more embodiments are set forth in the accompanying drawings and the description below. Other features and advantages will be apparent from the description and drawings, and from the claims.

FIG. 1 is an explanatory diagram illustrating a schematic configuration of a biological signal acquisition device according to a first embodiment.

FIG. 2 is a perspective view illustrating an external configuration of a sensor and a noise reduction member according to the first embodiment.

FIG. 3 is an explanatory diagram illustrating a relationship between a diameter of the noise reduction member, pressure, and noise amplitude.

FIG. 4 is a block diagram illustrating a schematic configuration of the biological signal acquisition device according to a second embodiment.

FIG. 5 is a block diagram illustrating a schematic configuration of the biological signal acquisition device according to a third embodiment.

FIG. 6 is a block diagram illustrating a schematic configuration of the biological signal acquisition device according to a fourth embodiment.

FIG. 7 is a block diagram illustrating a schematic configuration of the biological signal acquisition device according to a fifth embodiment.

FIG. 8 is a block diagram illustrating a schematic configuration of the biological signal acquisition device according to a sixth embodiment.

### DETAILED DESCRIPTION

To begin with, examples of relevant techniques will be described.

A biological signal acquisition device having sensors such as piezoelectric sensors provided in a seat of a vehicle, which acquires biological signals such as ballistic movements or ballistocardiograms of a heart of a person seated on the seat, has been proposed. In the biological signal acquisition device according to a comparative example, a recess is provided in a seat surface, a vibration-damping material is disposed at a bottom of the recess, a support member is placed on top of the vibration-damping material, and a piezoelectric sensor is further provided on top of the support member. As a result, a gap is provided between a lateral surface of the piezoelectric sensor and a lateral surface of the recess, and the vibration-damping material suppresses an input of vibration noise to the sensor via the seat by its damping action.

However, in the biological signal acquisition device of the comparative example, in order to shift a resonance frequency of the vibration-damping material to a lower frequency side and remove noise in a frequency band of biological signals of about 10 to 50 Hz, it is necessary to either use a softer material for the vibration-damping material or increase a mass of the vibration-damping material. If the vibration-damping material is made of a soft material, there is a risk that the material will be compressed by a force applied from a human body, thereby altering its physical properties. Therefore, it is necessary to increase a thickness of the vibration-damping material in a direction of vibration input. In addition, in order to increase the mass of the vibration-damping material, it is necessary to increase its density or volume.

In this way, with the configuration of the biological signal acquisition device in the comparative example, it is inevitable to increase the size of the vibration-damping material, resulting in an issue that the biological signal acquisition device becomes larger. In contrast to the comparative example, according to a biological signal acquisition device of the present disclosure, the biological signal acquisition device can be miniaturized.

According to one embodiment of the present disclosure, a biological signal acquisition device includes a sensor that detects a biological signal of a human body in a seated state where the human body is seated on a seat. The device has a noise reduction member disposed between the sensor and the seat, in contact with both the sensor and the seat, and the noise reduction member reduces noise input from the seat to the sensor. The device further includes at least one of (i) a circuit and (ii) a processor with a memory storing computer program code executable by the processor, and the at least one of the circuit and the processor obtains biological information using the biological signal detected by the sensor. An opposing direction is defined as a direction along which the sensor, the noise reduction member, and a contact portion of contact between the noise reduction member and the seat are aligned. A projected area of the noise reduction member is larger than a projected area of the sensor when viewed in the opposing direction, and a Young's modulus of the noise reduction member is higher than a Young's modulus of the contact portion of the seat.

According to this configuration, in the biological signal acquisition device, when viewed in the opposing direction, the projected area of the noise reduction member is larger than the projected area of the sensor. Therefore, pressure from vibration noise input through a portion of the sensor in contact with the noise reduction member can be reduced, compared to a configuration without the noise reduction member, due to the presence of the noise reduction member. As a result, force corresponding to vibration noise input to the sensor can be reduced. In addition, since the Young's modulus of the noise reduction member is higher than that of the contact portion of the seat back, when the vibration noise is input from the seat back to the noise reduction member, or when the pressing force from the human body is input to the noise reduction member via the sensor, it is possible to reduce the concentration of the force on the portion in contact with the sensor caused by deflection of portions of the noise reduction member other than the portion in contact with the sensor. As a result, the noise reduction effects of the noise reduction member on vibration noise can be improved.

According to another embodiment of the present disclosure, a biological signal acquisition device includes a sensor in contact with a seat, and the sensor detects, in a seated state where a human body is seated on the seat, a biological signal of the human body. The device has a noise reduction member in contact with the seat, and the noise reduction member reduces noise input from the seat to the sensor. The device further includes at least one of (i) a circuit and (ii) a processor with a memory storing computer program code executable by the processor, and the at least one of the circuit and the processor obtain biological information using the biological signal detected by the sensor. The sensor includes a lateral surface intersecting a contact surface of the sensor, and the contact surface is a surface that contacts the human body in the seated state. The noise reduction member is in contact with a part of the lateral surface of the sensor in a thickness direction of the sensor. A Young's modulus of the noise reduction member is higher than a Young's modulus of a portion of the seat including the contact surface. The Young's modulus of the noise reduction member is higher than the Young's modulus of the portion of the seat including the contact surface.

According to this configuration, the biological signal acquisition device is arranged in contact with the seat back, and is provided with the noise reduction member that is arranged in contact with the part of the thickness direction of the lateral surface of the sensor, which is the surface intersecting the contact surface of the sensor that comes into contact with the human body in the seated state, and serves to reduce the noise input from the seat back to the sensor. Therefore, compared to a comparative example configuration without the noise reduction member, the pressure due to the vibration noise input from the seat back to the sensor can be reduced. As a result, force corresponding to vibration noise input to the sensor can be reduced. In addition, since the Young's modulus of the noise reduction member is higher than that of the contact portion of the seat back, when the vibration noise from the seat back or the pressing force from the human body is input to the noise reduction member, it is possible to suppress force concentration on the sensor caused by the deformation of the noise reduction member. As a result, the noise reduction effects of the noise reduction member on vibration noise can be improved.

### First Embodiment

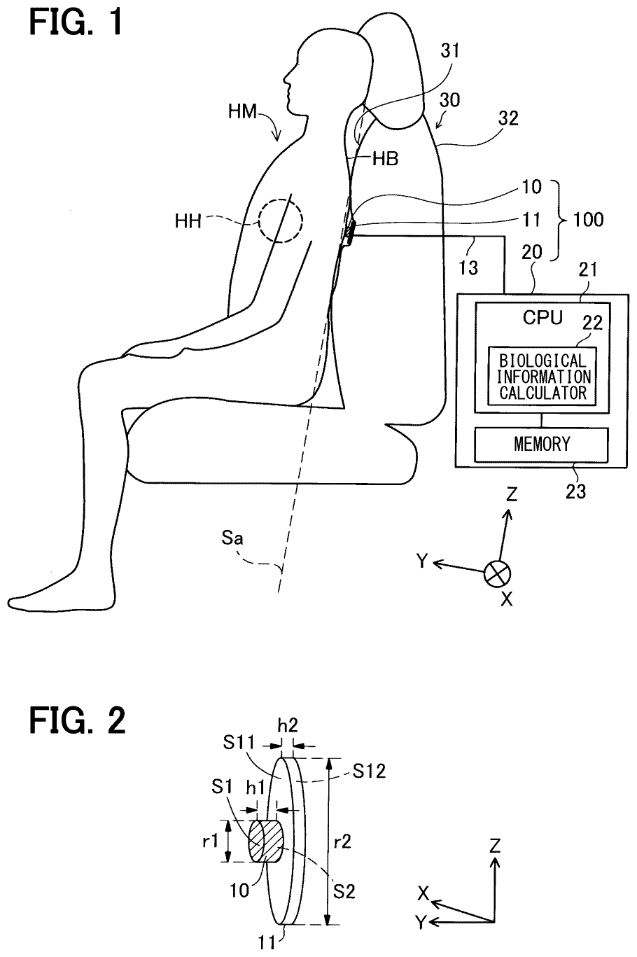

A biological signal acquisition device 100 according to a first embodiment shown in FIG. 1 is used mounted on a seat 30. The seat 30 has a seat cushion and a seat back. The biological signal acquisition device 100 acquires biological signals from a body HB of a person HM seated on the seat 30. In the present embodiment, the biological signals acquired by the biological signal acquisition device 100 are a ballistocardiogram of a heart HH of the person HM. It should be noted that, instead of the ballistocardiogram, any signal indicating vibrations generated by the person HM, such as respiration, pulse, heart sounds, organ movements, fetal movements, or body movements (bodily motion), may be acquired. It should be noted that the biological signal acquisition device 100 is configured to be capable of acquiring biological signals regardless of whether the person HM is wearing clothing during measurement.

In the present embodiment, the seat 30 is configured as a vehicle seat. The seat 30 is formed, for example, of a material that combines softness and resilience, such as polyurethane, and is made of a material with a relatively low Young's modulus. It should be noted that, instead of a vehicle seat, the seat may be configured as a seat for any moving objects such as a train or ship, or as a seat fixedly or movably arranged on any indoor or outdoor floor. All components of the biological signal acquisition device 100 may be mounted on the seat 30, or a part of the biological signal acquisition device 100 may be mounted on the seat 30.

FIG. 1 shows an X-axis, a Y-axis, and a Z-axis that are orthogonal to each other. The X-axis is an axis parallel to a width direction of the seat 30. The Z-axis is an axis that, in a state where a person HM is seated on the seat 30 (hereinafter referred to as the “seated state”), is parallel to a first surface Sa where the human body HB of the person HM and a front surface 31 of the seat back 30 are in contact, and is perpendicular to the X-axis. The Z-axis is parallel to a direction approximating a vertical direction. The Y-axis is an axis parallel to a facing direction. The facing direction refers to a direction in which a contact portion of the human body HB that is in contact with the seat back 30 in the seated state, that is, a back of the human body HB, faces the seat back 30. In the present embodiment, the Y-axis is parallel to a direction approximating a horizontal direction. It should be noted that an X-axis, a Y-axis, and a Z-axis in the other figures correspond to the X-axis, the Y-axis, and the Z-axis in FIG. 1. In the present embodiment, a positive X-direction and a negative X-direction are sometimes collectively referred to as an X-axis direction. Similarly, a positive Y-direction and a negative Y-direction are sometimes collectively referred to as a Y-axis direction, and a positive Z-direction and a negative Z-direction are sometimes collectively referred to as a Z-axis direction.

The biological signal acquisition device 100 includes a sensor 10, a noise reduction member 11, and a computer 20.

The sensor 10 detects a biological signal, specifically the ballistocardiogram, by coming into contact with the human body HB in the seated state. In the present embodiment, the sensor 10 is configured as a piezoelectric sensor. As shown in FIG. 2, in the present embodiment, the sensor 10 has a thin, cylindrical external shape. In the present embodiment, a diameter r1 of the sensor 10 is 10 mm (millimeters). A thickness h1 of the sensor 10 is 1 mm. As shown in FIG. 1, in the seated state, the sensor 10 comes into contact with the human body HB of the person HM, specifically with the back of the human. A thickness direction of the sensor 10 is parallel to the Y-axis direction. The sensor 10 is arranged such that both an end face S1 positioned at the positive Y-direction and an end face S2 positioned at the negative Y-direction of the sensor 10 are parallel to the first surface Sa.

The noise reduction member 11 is disposed between the sensor 10 and the seat back 30, in contact with both the sensor 10 and the seat back 30. The noise reduction member 11 is a member for reducing noise input to the sensor 10 from the seat back 30. The “noise input to the sensor 10 from the seat back 30” refers to vibrations occurring while the vehicle is in motion, such as road noise or vibration noise associated with engine rotation. As shown in FIG. 2, the noise reduction member 11 has a thin, disk-shaped external appearance. A diameter r2 of the noise reduction member 11 is 40 mm. A thickness h2 of the noise reduction member 11 is 0.5 mm. As shown in FIG. 2, the noise reduction member 11 is arranged such that both an end face S11 positioned at the positive Y-direction and an end face S12 positioned at the negative Y-direction of the noise reduction member 11 are parallel to the first surface Sa. The end face S11 of the noise reduction member 11 is in contact with the end face S2 of the sensor 10. As shown in FIG. 1, the end face S12 of the noise reduction member 11 is in contact with the front surface 31 of the seat back 30. In the present embodiment, the sensor 10 and the noise reduction member 11 are arranged in contact with each other such that their respective centers overlap in the Y-axis direction. In the present embodiment, the sensor 10 is bonded to the noise reduction member 11 with an adhesive. The noise reduction member 11 is formed from a material having a higher Young's modulus than the seat back 30. More specifically, in the present embodiment, the noise reduction member 11 is formed of brass. It should be noted that, instead of brass, the noise reduction member 11 may be formed from any material having a higher Young's modulus than polyurethane. For example, the noise reduction member 11 may be formed from a material having a hardness of 100 MPa (megapascals) or more.

As shown in FIG. 1, the computer 20 includes a CPU (Central Processing Unit) 21 and a memory 23. The computer 20 may be configured, for example, as a microcontroller. The CPU 21 functions as a biological information calculator 22 by executing programs stored in the memory 23. The biological information calculator 22 obtains biological information using the biological signals detected by the sensor 10. More specifically, the biological information calculator 22 obtains biological information such as heart rate and heart beat interval using the ballistocardiogram or the cardiac pulsation detected by the sensor 10. The computer 20 is electrically connected to the sensor 10 via a communication line 13. The computer 20 receives the biological signals detected by the sensor 10.

As shown in FIG. 2 and as previously described, when viewed in the opposing direction, that is, in the Y-axis direction, a projected area of the noise reduction member 11 is larger than a projected area of the sensor 10. Therefore, in the seated state shown in FIG. 1, a pressure of vibration noise input from the seat back 30 to the sensor 10 via the noise reduction member 11 is reduced compared to a configuration in which the noise reduction member 11 is absent and the sensor 10 is directly disposed on the front surface 31 of the seat back 30 (hereinafter referred to as a “comparative example configuration”). Since an area of the end face S2 of the sensor 10 is the same in both the present embodiment and the comparative example configuration, the force corresponding to the vibration noise input from the seat back 30 to the sensor 10 can be reduced in the present embodiment compared to the comparative configuration. Further, the Young's modulus of the noise reduction member 11 is higher than that of the contact portion of the seat back 30 where the noise reduction member 11 is in contact. Therefore, when a pressing force from the human body HB is input to the noise reduction member 11 via the sensor 10, it is possible to suppress portions of the noise reduction member 11 other than the portion in contact with the sensor 10 from deflecting, and to prevent concentration of force (such as reaction force of the pressing force or vibration noise force) on the portion of the noise reduction member 11 in contact with the sensor 10. As a result, a noise reduction effect on vibration noise provided by the noise reduction member 11 can be improved.

The projected area of the noise reduction member 11 and the sensor 10 refers to an area projected onto a plane having a perpendicular line parallel to the Y-axis, in other words, an area projected onto the X-Z plane, which is perpendicular to the opposing direction, by the noise reduction member 11 and the sensor 10.

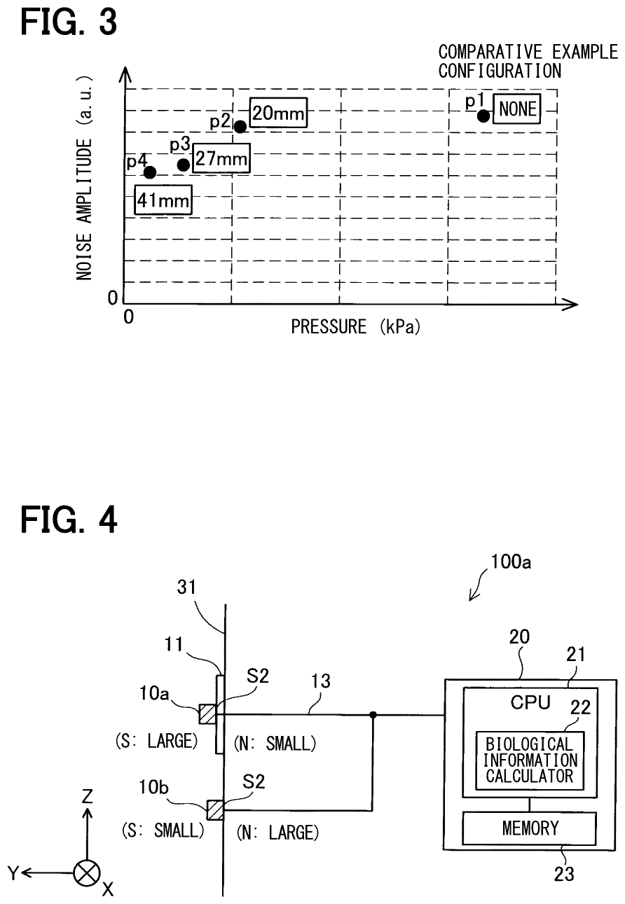

In FIG. 3, a horizontal axis indicates a pressure (kPa) at the end face S2 of the sensor 10, and a vertical axis indicates an amplitude of noise (arbitrary units) input to the sensor 10. As shown in FIG. 3, the magnitude (amplitude) of noise input to the sensor 10 can vary depending on a size of the diameter r2 of the noise reduction member 11. More specifically, in FIG. 3, in a configuration without the noise reduction member 11 (comparative example), the pressure at the end face S2 of the sensor 10 is relatively high, and a point p1 with a relatively large noise amplitude is obtained. Contrary to this, at point p2 where the diameter r2 is 20 mm, at point p3 where the diameter r2 is 27 mm, and at point p4 where the diameter r2 is 41 mm, the pressure and the noise amplitude are reduced progressively in this order. For example, the pressure at point p4 is one-third or less of the pressure at point p1, and the noise amplitude at point p4 is reduced to approximately two-thirds of the noise amplitude at point p1. As described above, in the biological signal acquisition device 100 of the present embodiment, by making the diameter r2 larger (four times) than the diameter r1, the noise amplitude can be significantly reduced compared to the configuration of the comparative example. Therefore, the diameter r2 may be of any size within a range indicated, for example, by the following equation (1).

1×r1<r2<5×r1 (1)

In addition, the diameter r2 is preferably of any size within a range indicated by the following equation (2).

1.5×r1<r2<4.5×r1 (2)

Furthermore, the diameter r2 is more preferably of any size within a range indicated by the following equation (3).

2×r1<r2<4×r1 (3)

According to the biological signal acquisition device 100 of the first embodiment described above, when viewed in the opposing direction (Y-axis direction), the projected area of the noise reduction member 11 is larger than the projected area of the sensor 10. Therefore, the pressure caused by vibration noise entering from the end face S2, which is in contact with the noise reduction member 11 in the sensor 10, can be reduced by the presence of the noise reduction member 11 compared to the comparative example configuration without the noise reduction member 11. As a result, the force corresponding to vibration noise input to the sensor 10 can be reduced. In addition, since the Young's modulus of the noise reduction member 11 is higher than that of the contact portion of the seat back 30, when the vibration noise is input from the seat back 30 to the noise reduction member 11, or when the pressing force from the human body HB is input to the noise reduction member 11 via the sensor 10, it is possible to reduce the concentration of the force on the portion in contact with the sensor 10 caused by deflection of portions of the noise reduction member 11 other than the portion in contact with the sensor 10. As a result, the noise reduction effect of the noise reduction member 11 on vibration noise can be improved.

### Second Embodiment

A biological signal acquisition device 100a of a second embodiment shown in FIG. 4 differs from the biological signal acquisition device 100 of the first embodiment shown in FIG. 1 in that it is equipped with sensors 10a and 10b. The other components of the biological signal acquisition device 100a of the second embodiment are the same as those of the biological signal acquisition device 100, and therefore identical components are denoted by the same reference numerals and detailed descriptions thereof are omitted.

Both the sensor 10a and the sensor 10b have the same configuration as the sensor 10 of the first embodiment. The sensor 10a is disposed in contact with the noise reduction member 11, as in the sensor 10 of the first embodiment. On the other hand, the sensor 10b is not in contact with the noise reduction member 11, but is instead disposed in direct contact with the front surface 31 of the seat back 30. The sensor 10a and the noise reduction member 11 have the same configuration as the sensor 10 and the noise reduction member 11 of the first embodiment. In the present embodiment, for convenience of explanation, the sensor 10a is referred to as a first sensor 10a, and the sensor 10b is referred to as a second sensor 10b. Both the first sensor 10a and the second sensor 10b are connected to the computer 20 via the communication line 13.

As shown in FIG. 4, the first sensor 10a is disposed in contact with the front surface 31 of the seat back 30 via the noise reduction member 11. On the other hand, the second sensor 10b is disposed in direct contact with the front surface 31 of the seat back 30 without the noise reduction member 11. Therefore, the end face S1 of the first sensor 10a is positioned further in the positive Y-direction than the end face S1 of the second sensor 10b. Accordingly, in the seated state, the first sensor 10a is pressed against the human body HB more strongly than the second sensor 10b. Therefore, in the seated state, the amplitude of the biological signal input to the first sensor 10a becomes larger than the amplitude of the biological signal input to the second sensor 10b.

On the other hand, since the first sensor 10a is disposed in contact with the noise reduction member 11, as in the sensor 10 of the first embodiment, the pressure on the negative Y-direction end face S2 is smaller than the pressure on the negative Y-direction end face S2 of the second sensor 10b. Therefore, the vibration noise input to the first sensor 10a from the seat 30 is reduced compared to the vibration noise input to the second sensor 10b.

In the present embodiment, the biological information calculator 22 calculates a heart rate and a heartbeat interval, using a differential signal between a signal including the biological signal detected by the first sensor 10a and a signal including the biological signal detected by the second sensor 10b. The term “signal including the biological signal” refers to a signal that includes both a signal indicative of the ballistocardiogram and a signal indicative of the vibration noise input from the seat back 30. More specifically, the biological information calculator 22 determines the heart rate using the differential signal (ΔS) between the ballistocardiogram signals detected by the first sensor 10a and the second sensor 10b, as well as the differential signal (ΔN) between the vibration noise signals detected by the first sensor 10a and the second sensor 10b.

As described above, the amplitude of the biological signal input to the first sensor 10a becomes larger than the amplitude of the biological signal input to the second sensor 10b. On the other hand, the vibration noise input to the first sensor 10a is reduced compared to the vibration noise input to the second sensor 10b. Here, assuming that a relationship between the biological signal (S) and vibration noise (N) input to the two sensors 10a and 10b is clear, a ratio of ΔS to ΔN (S/N ratio) is improved compared to a ratio of the biological signal (S) to the vibration noise (N) detected by the first sensor 10a. For example, when it is clear that the S/N ratio of the first sensor 10a is 10:5 and the S/N ratio of the second sensor 10b is 1:2, then ΔS:ΔN=9:3, which represents an improvement over 10:5.

The biological signal acquisition device 100a of the second embodiment described above provides the same effects as the biological signal acquisition device 100 of the first embodiment. In addition, since the biological information is obtained using the differential signal between the signal including the biological signal detected by the first sensor 10a, which is disposed in contact with the noise reduction member 11, and the signal including the biological signal detected by the second sensor 10b, which is not in contact with the noise reduction member 11 and is disposed in contact with the seat back 30, it is possible to obtain biological information based on a signal with improved S/N, thereby enabling highly accurate acquisition of the biological information.

### Third Embodiment

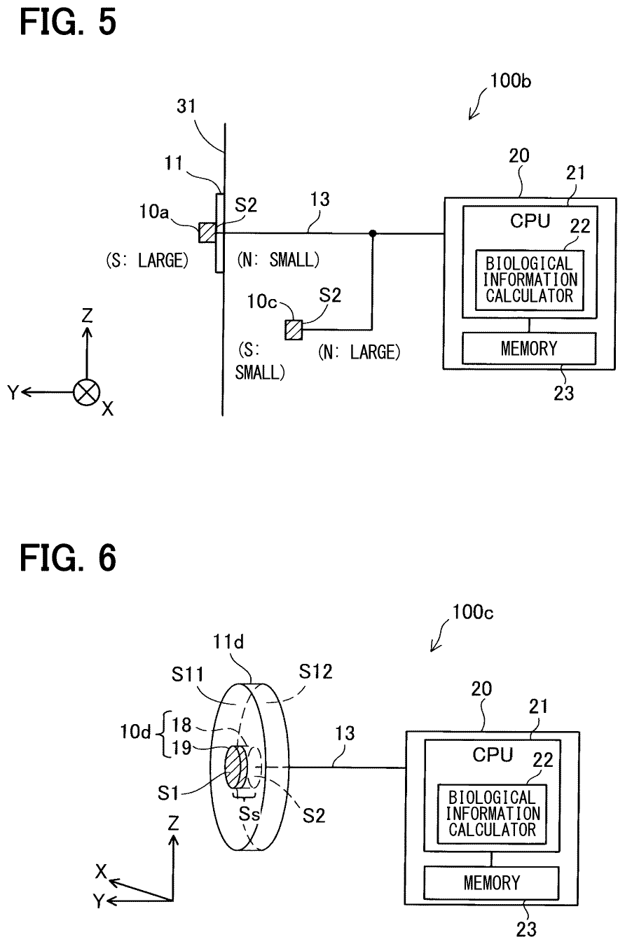

A biological signal acquisition device 100b of a third embodiment shown in FIG. 5 differs from the biological signal acquisition device 100 of the first embodiment shown in FIG. 1 in that it is provided with sensors 10a and 10c. The other components of the biological signal acquisition device 100b of the third embodiment are the same as those of the biological signal acquisition device 100, and therefore, identical reference numerals are used for identical components and detailed descriptions thereof are omitted.

Both the sensor 10a and the sensor 10b have the same configuration as the sensor 10 of the first embodiment. The sensor 10a is the same as the first sensor 10a of the second embodiment. That is, the sensor 10a is disposed in contact with the noise reduction member 11. The sensor 10c is not in contact with the noise reduction member 11 and is disposed inside the seat back 30. In the present embodiment, for convenience of explanation, the sensor 10a is referred to as a first sensor 10a, and the sensor 10c is referred to as a third sensor 10c. Both the first sensor 10a and the third sensor 10c are connected to the computer 20 via the communication line 13.

As shown in FIG. 5, the first sensor 10a is disposed in contact with the front surface 31 of the seat back 30 via the noise reduction member 11. On the other hand, the third sensor 10c is disposed in direct contact with the seat back 30 without the noise reduction member 11 interposed. In the seated state, the third sensor 10c is not in contact with the human body HB. Therefore, an amplitude of the biological signal input to the third sensor 10c is smaller than an amplitude of the biological signal input to the first sensor 10a, which is in direct contact with the human body HB.

On the other hand, since the first sensor 10a is disposed in contact with the noise reduction member 11, as in the sensor 10 of the first embodiment, the pressure on the negative Y-direction end face S2 is smaller than the pressure on the negative Y-direction end face S2 of the third sensor 10c. Therefore, the vibration noise input to the first sensor 10a from the seat 30 is reduced compared to the vibration noise input to the third sensor 10c.

In the present embodiment, the biological information calculator 22, as in the biological information calculator 22 of the second embodiment described above, determines the biological information such as a heart rate and a heart interval using a differential signal between a signal including the biological signal detected by the first sensor 10a and a signal including the biological signal detected by the third sensor 10c. Accordingly, in the biological signal acquisition device 100b of the third embodiment, as in the biological signal acquisition device 100a of the second embodiment, it is possible to improve the S/N ratio of the biological signal to vibration noise.

The biological signal acquisition device 100b of the third embodiment described above provides the same effects as the biological signal acquisition device 100 of the first embodiment. In addition, since the biological information is obtained using the differential signal between the signal including the biological signal detected by the first sensor 10a, which is arranged in contact with the noise reduction member 11, and the signal including the biological signal detected by the third sensor 10c, which is not in contact with the noise reduction member 11 and is arranged inside the seat back 30, it is possible to obtain the biological information based on a signal with improved S/N ratio, thereby allowing for highly accurate acquisition of the biological information.

### Fourth Embodiment

A biological signal acquisition device 100c according to a fourth embodiment shown in FIG. 6 differs from the biological signal acquisition device 100 of the first embodiment shown in FIG. 1 in that it is provided with a sensor 10d instead of the sensor 10, and a noise reduction member 11d instead of the noise reduction member 11. Since the other components of the biological signal acquisition device 100c of the fourth embodiment are the same as those of the biological signal acquisition device 100, identical reference numerals are given to identical components, and a detailed description thereof will be omitted.

A relative arrangement position of the sensor 10d with respect to the noise reduction member 11d is different from that of the sensor 10 in the first embodiment. A configuration of the sensor 10d, including its own size, is the same as that of the sensor 10 in the first embodiment. A portion of the sensor 10d in the thickness direction (Y-axis direction) is surrounded by and in contact with the noise reduction member 11d along the entire circumference of its lateral surface Ss. That is, the sensor 10d has a configuration in which a first portion 18, surrounded by the noise reduction member 11d, and a second portion 19, exposed from the noise reduction member 11d, are arranged in succession in the Y-axis direction. In other words, the noise reduction member 11d is arranged so as to surround, along the entire circumference of the sensor 10d, a portion of the lateral surface Ss of the sensor 10d in the thickness direction. A through-hole for accommodating the sensor 10d is provided at a center of the noise reduction member 11d, and a portion of the sensor 10d on the negative Y-direction side is accommodated in this through hole. The negative Y-direction end face S2 of the sensor 10d and the negative Y-direction end face S12 of the noise reduction member 11d are present on the same plane, and both are arranged in contact with the front surface 31 of the seat back 30. It can be said that the lateral surface Ss is a surface that intersects with the end face S1, which comes into contact with the human body HB.

With such a configuration, a pressure on the end face S2 of the sensor 10d is reduced compared to a comparative example configuration without the noise reduction member 11d. Therefore, the amplitude of vibration noise input from the end face S2 of the sensor 10d can be reduced, as in the first embodiment.

The biological signal acquisition device 100c of the fourth embodiment described above is arranged in contact with the seat back 30, and is provided with the noise reduction member 11d that is arranged in contact with a part of the thickness direction of the lateral surface Ss of the sensor 10d, which is the surface intersecting the contact surface (end face S1) of the sensor 10d that comes into contact with the human body HB in the seated state, and serves to reduce the noise input from the seat back 30 to the sensor 10d. Therefore, compared to the comparative example configuration without the noise reduction member 11d, the pressure due to the vibration noise input from the seat back 30 to the sensor 10d can be reduced. As a result, the force corresponding to vibration noise input to the sensor 10d can be reduced. In addition, since the Young's modulus of the noise reduction member 11d is higher than that of the contact portion of the seat back 30, when the vibration noise from the seat back 30 or the pressing force from the human body HB is input to the noise reduction member 11d, it is possible to suppress force concentration on the sensor 10d caused by the deformation of the noise reduction member 11d. As a result, the noise reduction effect of the noise reduction member 11d on vibration noise can be improved.

Furthermore, since the noise reduction member 11d is arranged so as to surround a part of the thickness direction of the lateral surface Ss of the sensor 10d around the entire periphery of the sensor 10d, compared to a configuration in which the noise reduction member 11d surrounds only a portion of the periphery of the sensor 10d in the thickness direction of the lateral surface Ss, the pressure due to vibration noise can be reduced even further.

### Fifth Embodiment

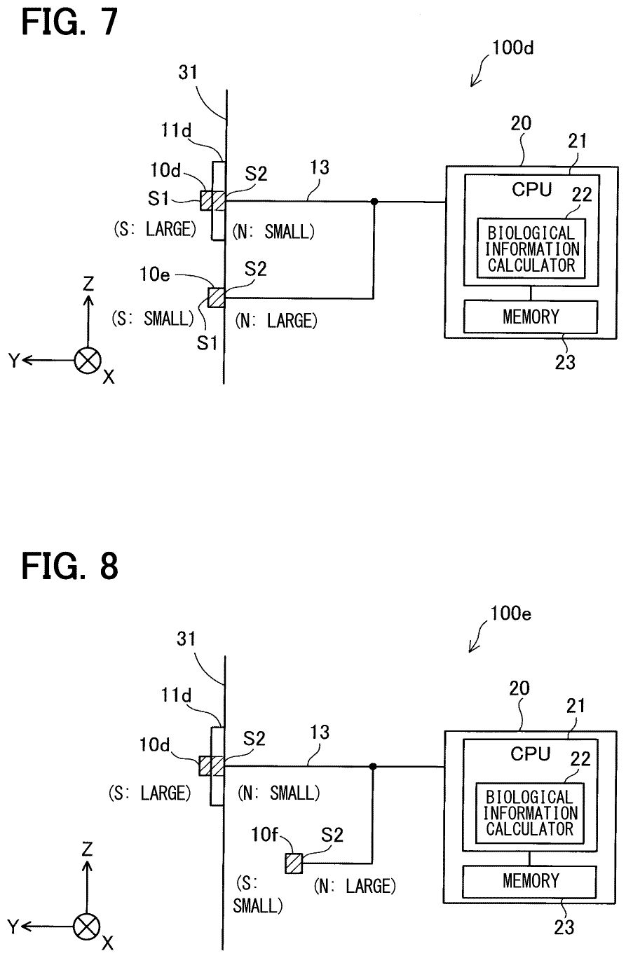

A biological signal acquisition device 100d of a fifth embodiment shown in FIG. 7 differs from the biological signal acquisition device 100c of the fourth embodiment shown in FIG. 6 in that it is equipped with sensors 10d and 10e. The other configurations of the biological signal acquisition device 100d of the fifth embodiment are the same as those of the biological signal acquisition device 100c, and therefore, identical components are denoted by the same reference numerals and detailed descriptions thereof are omitted.

Both the sensor 10d and the sensor 10e have the same configuration as the sensor 10d of the fourth embodiment. The sensor 10d, like the sensor 10d of the fourth embodiment, is disposed in contact with a noise reduction member 11d. On the other hand, the sensor 10e is not in contact with the noise reduction member 11d, but is disposed in direct contact with the front surface 31 of the seat back 30. The sensor 10d and the noise reduction member 11d have the same configuration as the sensor 10d and the noise reduction member 11d of the fourth embodiment. In the present embodiment, for convenience of explanation, the sensor 10d is referred to as a first sensor 10d, and the sensor 10e is referred to as a second sensor 10e. Both the first sensor 10d and the second sensor 10e are connected to the computer 20 via the communication line 13.

As shown in FIG. 7, the first sensor 10d is disposed in contact with the front surface 31 via the noise reduction member 11d. On the other hand, the second sensor 10e is disposed in direct contact with the front surface 31 without the noise reduction member 11d. Therefore, the end face S1 of the first sensor 10d is positioned further in the positive Y-direction than the end face S1 of the second sensor 10e. Accordingly, in the seated state, the first sensor 10d is pressed against the human body HB more strongly than the second sensor 10e. Therefore, in the seated state, the amplitude of the biological signal input to the first sensor 10d becomes larger than the amplitude of the biological signal input to the second sensor 10e.

On the other hand, since the first sensor 10d is disposed in contact with the noise reduction member 11d, as in the sensor 10d of the fourth embodiment, the pressure on the negative Y-direction end face S2 is smaller than the pressure on the negative Y-direction end face S2 of the second sensor 10e. Therefore, the vibration noise input from the seat back 30 to the first sensor 10d is reduced compared to the vibration noise input to the second sensor 10e.

In the present embodiment, as the second embodiment, the biological information calculator 22 calculates a heart rate and a heart beat interval, using a differential signal between a signal including the biological signal detected by the first sensor 10d and a signal including the biological signal detected by the second sensor 10e. Accordingly, in the biological signal acquisition device 100d of the fifth embodiment, as in the biological signal acquisition device 100a of the second embodiment, it is possible to improve the S/N ratio of the biological signal to vibration noise.

The biological signal acquisition device 100d of the fifth embodiment described above provides effects similar to those of the biological signal acquisition device 100c of the fourth embodiment. In addition, since the biological information is obtained using the differential signal between the signal including the biological signal detected by the first sensor 10d, which is disposed in contact with the noise reduction member 11d, and the signal including the biological signal detected by the second sensor 10e, which is not in contact with the noise reduction member 11d and is disposed in contact with the seat back 30, it is possible to obtain biological information based on a signal with improved S/N, thereby enabling highly accurate acquisition of the biological information.

### Sixth Embodiment

A biological signal acquisition device 100e according to a sixth embodiment, shown in FIG. 8, differs from the biological signal acquisition device 100c of the fourth embodiment, shown in FIG. 6, in that it is provided with sensors 10d and 10f. The other components of the biological signal acquisition device 100e of the sixth embodiment are the same as those of the biological signal acquisition device 100c. Accordingly, identical reference numerals are used for identical components, and detailed explanations thereof are omitted.

Both the sensor 10d and the sensor 10f have the same configuration as the sensor 10d of the fourth embodiment. The sensor 10d is the same as the first sensor 10d of the fifth embodiment. That is, the sensor 10d is disposed in contact with the noise reduction member 11d. The sensor 10f is not in contact with the noise reduction member 11d and is disposed inside the seat back 30. In the present embodiment, for convenience of explanation, the sensor 10d is referred to as a first sensor 10d, and the sensor 10f is referred to as a third sensor 10f. Both the first sensor 10d and the third sensor 10f are connected to the computer 20 via the communication line 13.

As shown in FIG. 8, the first sensor 10d is disposed in contact with the front surface 31 of the seat back 30 via the noise reduction member 11d. On the other hand, the third sensor 10f is disposed in direct contact with the seat back 30 without the interposition of the noise reduction member 11d. In the seated state, the third sensor 10f is not in contact with the human body HB. Therefore, an amplitude of the biological signal input to the third sensor 10f is smaller than an amplitude of the biological signal input to the first sensor 10d, which is in direct contact with the human body HB.

On the other hand, since the first sensor 10d is disposed in contact with the noise reduction member 11d, as in the sensor 10d of the fourth embodiment, the pressure on the negative Y-direction end face S2 is smaller than the pressure on the negative Y-direction end face S2 of the third sensor 10f. Therefore, the vibration noise input from the seat back 30 to the first sensor 10d is reduced compared to the vibration noise input to the third sensor 10f.

In the present embodiment, the biological information calculator 22, as in the biological information calculator 22 of the fifth embodiment described above, determines the biological information such as a heart rate and a heart interval using a differential signal between a signal including the biological signal detected by the first sensor 10d and a signal including the biological signal detected by the third sensor 10f. Accordingly, in the biological signal acquisition device 100e of the sixth embodiment, as in the biological signal acquisition device 100d of the fifth embodiment, it is possible to improve the S/N ratio of the biological signal to vibration noise.

The biological signal acquisition device 100e of the sixth embodiment described above provides effects similar to those of the biological signal acquisition device 100c of the fourth embodiment. In addition, since the biological information is obtained using the differential signal between the signal including the biological signal detected by the first sensor 10d, which is arranged in contact with the noise reduction member 11d, and the signal including the biological signal detected by the third sensor 10f, which is not in contact with the noise reduction member 11d and is arranged inside the seat back 30, it is possible to obtain the biological information based on a signal with improved S/N ratio, thereby allowing for highly accurate acquisition of the biological information.

### OTHER EMBODIMENTS

The specific dimensions of the sensors 10, 10a to 10f and the noise reduction members 11, 11d in each embodiment are not limited to the values specifically described in each embodiment. For example, the diameter r1 of the sensors 10 and 10a to 10f is not limited to 10 mm. It may be any value smaller than diameter r2 and within a range of 0.1 mm to 500 mm. In addition, the thickness h1 of the sensors 10 and 10a to 10f may be any value within a range of 0.1 mm to 100 mm. Furthermore, the diameter r2 of the noise reduction members 11 and 11d may be any value larger than the diameter r1.

The specific shapes of the sensors 10, 10a to 10f and the noise reduction members 11, 11d in each embodiment are not limited to the shapes specifically described in each embodiment. For example, the sensors 10 and 10a to 10f may have a prismatic shape instead of a cylindrical shape. Similarly, the noise reduction members 11 and 11d may have a thin rectangular plate-like external shape instead of a disk shape.

In the fourth to sixth embodiments, the noise reduction member 11d is arranged so as to surround a part of the lateral surface Ss of the sensors 10d to 10f over the entire circumference of the sensor 10d. However, the present disclosure is not limited thereto. The noise reduction member 11d may be arranged so as to surround (contact) only a portion of the lateral surface Ss of the sensors 10d to 10f, that is, only a part of the entire circumference of the sensor 10d. Even with such a configuration, the presence of the noise reduction member 11d can reduce the pressure on the negative Y-direction end surface S2 of the sensors 10d to 10f.

In the fourth to sixth embodiments, when viewed in the opposing direction (Y-axis direction), the projected area of the noise reduction member 11d may be equal to or less than the projected area of the sensor 10d. Even with such a configuration, by arranging the noise reduction member 11d in contact with at least a part of the lateral surface Ss and at least a portion of the entire circumference of the sensor 10d, it is possible to reduce the pressure on the end surface S2 of the sensor 10d and to reduce the amplitude of vibration noise input to the sensor 10d.

The biological signal acquisition devices 100, 100a to 100e in each embodiment are merely examples, and various modifications are possible. For example, in the second, third, fifth, and sixth embodiments, the number of sensors is two, but it is not limited to two and may be any number of three or more. In addition, instead of being disposed on or inside the front surface 31 of the seat back 30, or in addition to being disposed on or inside the front surface 31 of the seat back 30, the sensors 10, 10a to 10f may also be disposed on a surface of a seat cushion inside the seat cushion.

The biological information calculator 22 and methods thereof described in the present disclosure may be implemented by a dedicated computer provided by configuring a processor and a memory programmed to execute one or more functions embodied by a computer program. Alternatively, the biological information calculator 22 and methods thereof described in the present disclosure may be implemented by a dedicated computer provided by configuring the processor with one or more dedicated hardware logic circuits. Alternatively, the biological information calculator 22 and methods thereof described in the present disclosure may be implemented by one or more dedicated computers configured with a combination of a processor and a memory programmed to execute one or more functions, and a processor configured with one or more hardware logic circuits. Furthermore, the computer program may be stored as instructions executed by a computer on a computer-readable non-transitory tangible recording medium.

While the present disclosure has been described with reference to embodiments thereof, it is to be understood that the disclosure is not limited to the embodiments and constructions. To the contrary, the present disclosure is intended to cover various modification and equivalent arrangements. In addition, while the various elements are shown in various combinations and configurations, which are exemplary, other combinations and configurations, including more, less or only a single element, are also within the spirit and scope of the present disclosure.

What is claimed is:

1. A biological signal acquisition device comprising:
a sensor configured to detect a biological signal of a human body in a seated state in which the human body is seated on a seat;
a noise reduction member disposed between the sensor and the seat, in contact with both the sensor and the seat, and configured to reduce noise input from the seat to the sensor; and
at least one of (i) a circuit and (ii) a processor with a memory storing computer program code executable by the processor, the at least one of the circuit and the processor configured to obtain biological information using the biological signal detected by the sensor, wherein
an opposing direction is a direction along which the sensor, the noise reduction member, and a contact portion between the noise reduction member and the seat are aligned,
a projected area of the noise reduction member is larger than a projected area of the sensor when viewed in the opposing direction, and
a Young's modulus of the noise reduction member is higher than a Young's modulus of the contact portion of the seat.

2. The biological signal acquisition device according to claim 1, wherein
the sensor is one of sensors,
the sensors includes:
a first sensor in contact with the noise reduction member; and
a second sensor in contact with the seat and not in contact with the noise reduction member, and
the at least one of the circuit and the processor is configured to obtain the biological information using a differential signal between a signal including the biological signal detected by the first sensor and a signal including the biological signal detected by the second sensor.

3. The biological signal acquisition device according to claim 1, wherein
the sensor is one of sensors,
the sensors includes:
a first sensor in contact with the noise reduction member; and
a third sensor disposed inside the seat and not in contact with the noise reduction member, and
the at least one of the circuit and the processor is configured to obtain the biological information using a differential signal between a signal including the biological signal detected by the first sensor and a signal including the biological signal detected by the third sensor.

4. A biological signal acquisition device comprising:
a sensor in contact with a seat and configured to detect, in a seated state in which a human body is seated on the seat, a biological signal of the human body;
a noise reduction member in contact with the seat and configured to reduce noise input from the seat to the sensor; and
at least one of (i) a circuit and (ii) a processor with a memory storing computer program code executable by the processor, the at least one of the circuit and the processor configured to obtain biological information using the biological signal detected by the sensor, wherein
the sensor includes a lateral surface intersecting a contact surface of the sensor, the contact surface being a surface that contacts the human body in the seated state,
the noise reduction member is in contact with a part of the lateral surface of the sensor in a thickness direction of the sensor, and
a Young's modulus of the noise reduction member is higher than a Young's modulus of a portion of the seat including the contact surface.

5. The biological signal acquisition device according to claim 4, wherein
the noise reduction member is disposed to surround, along an entire periphery of the sensor, the part of the lateral surface of the sensor in the thickness direction.

6. The biological signal acquisition device according to claim 4, wherein
the sensor is one of sensors,
the sensors includes:
a first sensor in contact with the noise reduction member; and
a second sensor in contact with the seat and not in contact with the noise reduction member, and
the at least one of the circuit and the processor is configured to obtain the biological information using a differential signal between a signal including the biological signal detected by the first sensor and a signal including the biological signal detected by the second sensor.

7. The biological signal acquisition device according to claim 4, wherein
the sensor is one of sensors,
the sensors includes:
a first sensor in contact with the noise reduction member; and
a third sensor disposed inside the seat and not in contact with the noise reduction member, and
the at least one of the circuit and the processor is configured to obtain the biological information using a differential signal between a signal including the biological signal detected by the first sensor and a signal including the biological signal detected by the third sensor.
